(19) United States
(12) Patent Application Publication
(10) Pub. No.: US 2026/0131137 A1
(43) Pub. Date: May 14, 2026

(54) MICROCURRENT ION PATCH INCLUDING AN ELECTROCHROMIC UNIT

(71) Applicant: SEOUL NATIONAL UNIVERSITY R&DB FOUNDATION, Seoul (KR)

(72) Inventors: Seung Kyun KANG, Seoul (KR); Sung Geun CHOI, Seoul (KR); Se Hun KANG, Seoul (KR)

(73) Assignee: SEOUL NATIONAL UNIVERSITY R&DB FOUNDATION, Seoul (KR)

(21) Appl. No.: 19/121,650

(22) PCT Filed: Oct. 18, 2023
(86) PCT No.: PCT/KR2023/016207

§ 371 Date: Apr. 16, 2025

(30) Foreign Application Priority Data
Oct. 18, 2022 (KR) 10-2022-0134100
Oct. 18, 2023 (KR) 10-2023-0139951

(51) Int. Cl. A61N 1/04; A61L 31/14; A61N 1/32

(52) U.S. Cl. CPC A61N 1/0428; A61L 31/145; A61N 1/0496; A61N 1/325

(57) ABSTRACT

Embodiments of the present invention relate to a microcurrent ion patch comprising an electrochromic unit, which allows users to visually confirm the usage state. A microcurrent ion patch comprising an electrochromic unit according to an embodiment of the present invention includes an iontophoresis ion patch and an electrochromic unit electrically connected to both electrodes of the ion patch. The electrochromic unit may comprise: a base thin film; an electrochromic material layer formed on the base thin film; and a hydrogel electrolyte layer formed on the electrochromic material layer.

### TECHNICAL FIELD

The present invention relates to a microcurrent ion patch. More specifically, the present invention relates to a microcurrent ion patch equipped with an electrochromic function.

### BACKGROUND ART

Recently, as people have become increasingly interested in cosmetic procedures, microcurrent ion patches using iontophoresis have gained attention as a more effective and certain method.

Conventional methods for delivering drugs to the human body have utilized injections or oral administration. However, injections are painful and difficult for the general public to use. Additionally, oral administration has the disadvantage of being indirect, slow, and difficult to control accurately.

On the other hand, the iontophoresis method increases the skin permeability of ionic drugs by applying a potential difference to the skin, thereby changing the electrical environment of the skin. Therefore, the iontophoresis method has the advantage of delivering drugs into the body in a simple and painless manner. Most of the existing patches using the iontophoresis method utilize the principle of a galvanic cell, where a reaction occurs spontaneously when the circuit is connected.

However, despite these advantages, conventional iontophoresis methods still have issues. It is difficult to determine whether the current is flowing properly and whether the drug is being delivered during the procedure. Additionally, users find it hard to easily check how long the device has been in use. These problems have made it difficult for users to trust the product, hindering commercialization.

### DETAILED DESCRIPTION OF THE INVENTION

### Technical Problem

The object of the present invention is to propose a microcurrent ion patch that allows the user to verify whether the current and drug are being effectively delivered without the need for separate measurement equipment.

Additionally, the object of the present invention is to enable the user to visually confirm not only whether the drug is being delivered but also the appropriate usage time of the patch, thus preventing side effects due to overuse of the ion patch.

Furthermore, the object of the present invention is to provide a microcurrent ion patch that can be disposed of in an environmentally friendly manner while being used as a disposable product.

### Technical Solution

The embodiments of the present invention relate to a microcurrent ion patch that includes an electrochromic unit, allowing users to visually confirm the status of use. To achieve this, the inventors applied the principle of electrochromism to an ion patch that delivers drugs beneficial for skin improvement, such as those for skin care and whitening, through electrochemical transdermal delivery.

The microcurrent ion patch including an electrochromic unit according to an embodiment of the present invention includes an iontophoresis ion patch and an electrochromic unit electrically connected to both electrodes of the ion patch. The electrochromic unit may include a base thin film; an electrochromic material layer formed on the base thin film; and a hydrogel electrolyte layer formed on the electrochromic material layer.

According to an embodiment, the electrochromic unit receives electrons from one electrode of the ion patch and provides electrons to the other electrode of the ion patch.

According to an embodiment, the electrochromic material layer of the electrochromic unit undergoes a reduction reaction due to the received electrons, resulting in electrochromism.

According to an embodiment, the electrochromic unit may have an increasing area of color change corresponding to the amount of drug delivered by the iontophoresis ion patch.

According to an embodiment, the electrochromic material layer includes spaced metal parts, and the oxide of the metal is formed between the metal parts.

According to an embodiment, the metal parts include tungsten (W), and the electrochromic material layer includes tungsten trioxide (WO₃).

According to an embodiment, the electrolyte layer may cause a redox reaction between ferrocyanide/ferrocyanide (Fe(CN)₆⁴⁻/Fe(CN)₆³⁻).

According to an embodiment, the ion patch may further include a plate-shaped frame; an electrode layer including electrodes that are stacked and spaced apart on the frame; and a gel layer stacked on each of the electrodes.

According to an embodiment, the electrode layer includes an anode containing magnesium (Mg) and a cathode containing molybdenum trioxide (MoO₃), and the gel layer may include one or more substances selected from the group consisting of alginate, gelatin, agar, maltose, and glycerol.

According to an embodiment, the frame may be made of PLGA (polylactic co-glycolic acid) or PGLA (N,N′-diacetylbacillosaminyl-diphospho-undecaprenol alpha-1,3-N-acetylgalactosaminyltransferase).

According to an embodiment, both the iontophoresis ion patch and the electrochromic unit may be made of biodegradable materials.

According to an embodiment, a resistor arranged in series between the electrochromic unit and the iontophoresis ion patch may further be included.

According to an embodiment, the electrochromic unit may consist of a plurality of units arranged in rows, connected in series.

According to an embodiment, the rows may be multiple, and the iontophoresis ion patch may be connected in a parallel structure, forming a heat configuration with each other.

The method of manufacturing a microcurrent ion patch including an electrochromic unit according to another embodiment of the present invention may include the steps of: preparing an electrochromic unit that includes an electrochromic material layer; preparing an ion patch that includes an anode and a cathode; and electrically connecting the ion patch and the electrochromic unit.

According to an embodiment, the step of electrically connecting may include connecting one end of the electrochromic material layer to the anode of the ion patch and connecting the other end of the electrochromic material layer to the cathode of the ion patch, so that the electrons from the ion patch pass through the electrochromic unit and return to the ion patch.

According to an embodiment, the step of preparing the electrochromic unit may include the steps of: forming an electrochromic material layer on the base thin film; and forming a hydrogel electrolyte layer on the electrochromic material layer.

According to an embodiment, the step of preparing the ion patch may include: an electrode layer formation step, where electrodes are stacked on a plate-shaped frame; and a gel layer formation step, where a gel is stacked on the electrodes.

### Effects of the Invention

The biodegradable microcurrent ion patch proposed in the present invention introduces a sequential color change function according to the amount of drug delivered, which visually informs the patient of the delivery amount. As a result, users can check, without the need for separate measurement equipment, how much of the drug has been delivered and how much more needs to be delivered.

Furthermore, by adjusting the number of units that undergo the electrochromic reaction based on the reaction time, users can easily adhere to the proper usage time of cosmetics, ensuring effective use and preventing side effects caused by overuse.

Additionally, since the electrochromic reaction is induced by the current flowing between the two electrodes of the conventional ion patch system, there is the advantage that no additional power, such as from a battery, is required.

### BRIEF DESCRIPTION OF THE DRAWINGS

FIG. 1 is a schematic diagram showing the electrochromic unit applied to the microcurrent ion patch according to an embodiment of the present invention, connected to a battery.

FIG. 2 is an experimental image showing the reaction current graph and the color change development speed over time for the electrochromic unit of FIG. 1.

FIG. 3 is a circuit diagram showing the electrochromic unit according to an embodiment of the present invention, connected in series with an iontophoresis ion patch and with an electrical resistor in between.

FIG. 4 is a voltage-time graph of the microcurrent ion patch including the electrochromic unit of FIG. 3.

FIG. 5 is an experimental image showing the color change amount over time for the microcurrent ion patch including the electrochromic unit of FIG. 3.

FIG. 6 is a schematic diagram showing the structure of the iontophoresis ion patch according to an embodiment of the present invention.

FIG. 7 is a schematic diagram showing the reaction that occurs when the electrochromic unit is electrically connected to the iontophoresis ion patch according to an embodiment of the present invention.

FIG. 8 is a schematic diagram showing the mechanism in which the degree of color change is controlled in accordance with the amount of drug delivered when the electrochromic unit is electrically connected to the iontophoresis ion patch according to an embodiment of the present invention.

FIG. 9 is a schematic diagram showing a structure in which a row formed by a plurality of electrochromic units is parallelly connected to the iontophoresis ion patch, with a resistor included between each row, represented in a plan view.

FIG. 10 is a schematic diagram showing a structure in which a row formed by a plurality of electrochromic units is parallelly connected to the iontophoresis ion patch, with a resistor included between each row, shown in a three-dimensional view including a stacked structure.

### MODE FOR CARRYING OUT THE INVENTION

The embodiments of the present invention are provided by way of example for the purpose of illustrating the technical spirit of the present invention. The scope of rights according to the present invention is not limited to the embodiments set forth below or the detailed descriptions thereof.

All technical and scientific terms used in the present invention, unless otherwise defined, have meanings that are generally understood by those of ordinary skill in the art to which the invention pertains. All terms used herein are selected for the purpose of more clearly describing the present invention and are not intended to limit the scope of rights of the present invention.

Expressions such as “comprising”, “including”, and “having” as used in the present invention shall be interpreted as open-ended terms that allow for the inclusion of other embodiments, unless otherwise stated in the phrases or sentences in which they appear.

The materials disclosed in the present invention are merely one example tested in a laboratory for experimental purposes, and the technical concept proposed by the inventors is not limited to specific materials but is directed to the principle itself of incorporating an electrochromic system into an ion patch.

According to an embodiment of the present invention, a microcurrent ion patch comprising an electrochromic unit includes an iontophoresis ion patch and an electrochromic unit electrically connected to both electrodes of the ion patch,

wherein the electrochromic unit may comprise: a base thin film; an electrochromic material layer formed on the base thin film; and a hydrogel electrolyte layer formed on the electrochromic material layer.

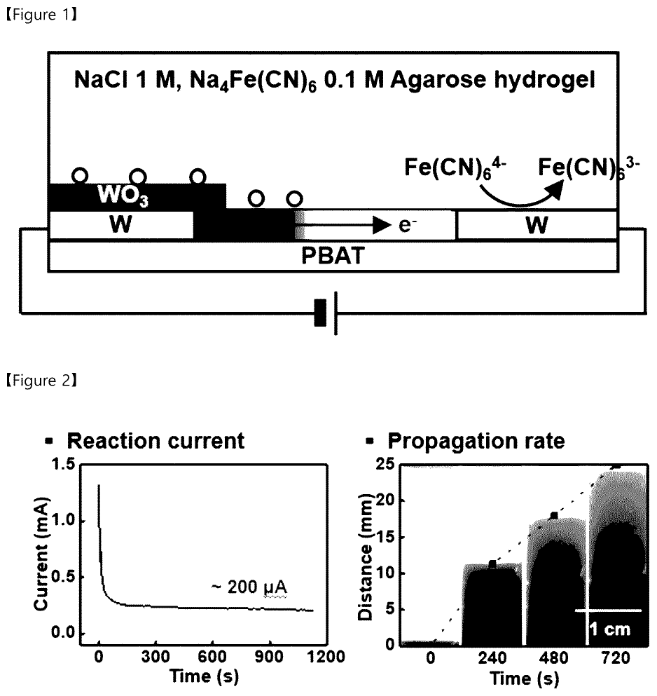

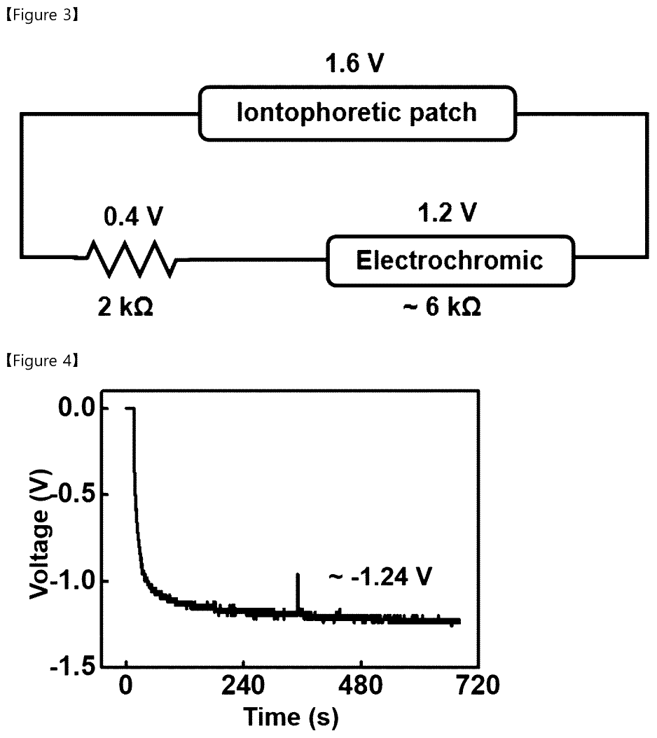

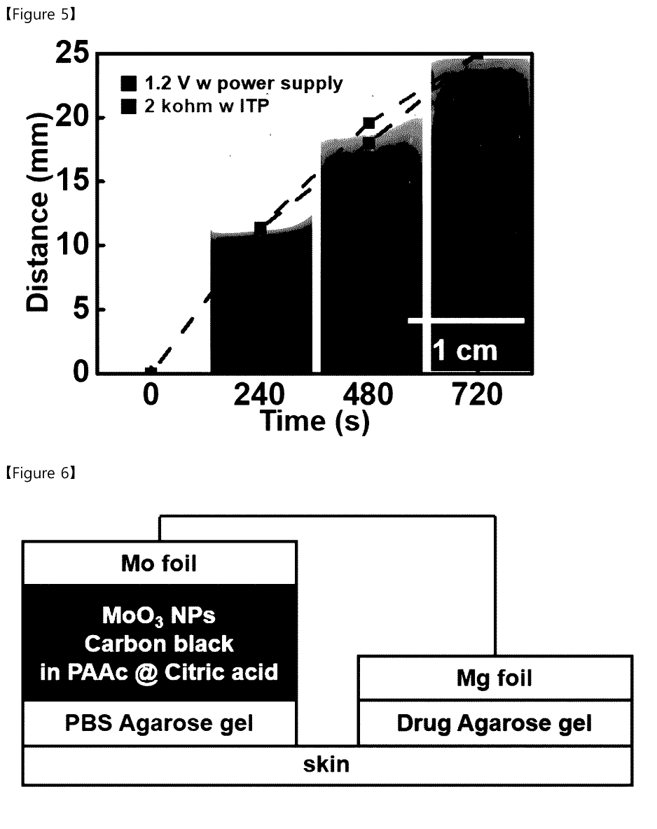

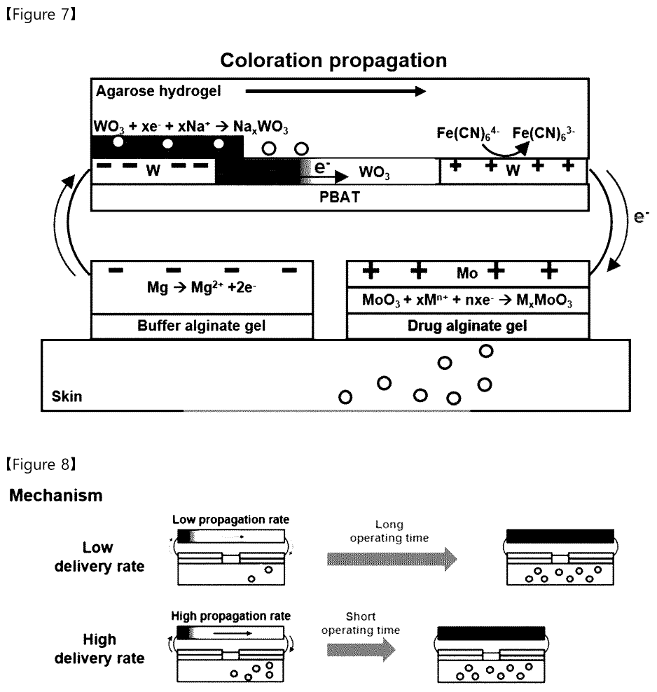

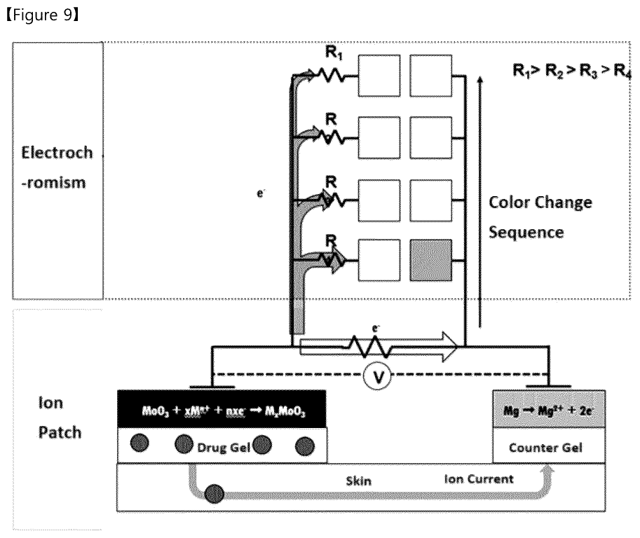

Hereinafter, with reference to FIGS. 1 to 10 described above, the structure and principle of the embodiments of the present invention will be described in detail.

A microcurrent ion patch comprising an electrochromic unit according to an embodiment of the present invention utilizes a structure in which, when a galvanic battery included in the iontophoresis ion patch is activated and a potential difference occurs between the metal electrodes of the anode and the cathode, the anode is oxidized to release electrons, and the cathode is reduced by accepting electrons.

By connecting both electrodes of such an iontophoresis ion patch to the electrochromic unit, the iontophoresis ion patch may function as a battery for the electrochromic unit.

The iontophoresis ion patch delivers a drug into the body using an electric field generated by the potential difference. The inventors of the present invention have completed the invention by introducing the potential difference and the flow of electrons, which are generated in the battery reaction of the ion patch, into the electrochromic system.

By using this method, as the discharge time of the battery continues, the drug is delivered by the electric field generated, and simultaneously, the electrochromic reaction occurs, thereby enabling the degree of drug delivery to be visualized through the electrochromic system without requiring a separate device.

Electrochromism, in this context, refers to a phenomenon in which the color changes depending on the oxidation or reduction state caused by an electrochemical reaction. In an embodiment of the present invention, tungsten trioxide (WO₃) is used as the material for the electrochromic material layer.

When a negative (−) electrode is applied to the electrochromic material layer, a reduction reaction occurs as electrons are supplied and cations in the electrolyte are inserted into the tungsten trioxide along the electric field formed in the electrolyte.

Under the above embodiment, the electrochromic material layer may be formed on a prepared thin film without introducing a separate electrode on the lower side.

By adopting such an electrode-free structure, the onset time of the electrochromic reaction can be delayed.

In conventional electrochromic systems, the time required for the full reaction is typically only a few seconds.

In contrast, the operating time of an iontophoresis ion patch generally ranges from approximately 10 minutes to several days.

In the present invention, the electrochromic response speed of the electrochromic system connected to the iontophoresis ion patch can be intentionally controlled using the aforementioned method.

According to one embodiment, the electrochromic unit may receive electrons from one electrode of the ion patch and supply the electrons to the other electrode of the ion patch. According to one embodiment, the electrochromic material layer of the electrochromic unit may undergo a reduction reaction due to the supplied electrons, thereby causing electrochromism.

According to one embodiment, the electrochromic unit may exhibit an increase in the area showing color change corresponding to the amount of drug delivered by the iontophoresis ion patch.

In one embodiment of the present invention, the electrochromic material layer can be designed to gradually propagate the electrochromic reaction laterally over time, based on the fact that the electrochromic material layer becomes partially conductive as the electrochromic reaction occurs without electrodes.

Through this, it is possible to implement a color change that spreads gradually, rather than a uniform color change over the entire area.

Since the electrochromic reaction proceeds more rapidly when the battery reaction is more active, the embodiments of the present invention enable not only the visualization of a particular state, but also the visualization of the degree of change in that state.

According to one embodiment, the electrochromic material layer may include spaced-apart metal portions and may include an oxide of the metal forming the metal portions, which is formed by filling the space between the metal portions.

According to one embodiment, the metal portions may include tungsten, and the electrochromic material layer may include tungsten trioxide (WO₃).

The metal portions may include a mesh structure of metal nanowires. In one example, the metal nanowires may have a radius ranging from 0.1 μm to several μm.

According to one embodiment, the electrolyte layer may undergo a redox reaction between ferrocyanide and ferricyanide (Fe(CN)₆⁴⁻/Fe(CN)₆³⁻).

In one embodiment, by selecting appropriate biodegradable materials for the anode and cathode used in the iontophoresis ion patch, the standard reduction potential difference may be designed to be approximately 1.2 V. In this case, ferricyanide ions may be used at the (+) electrode to induce an electrochromic reaction within this voltage range. Ferricyanide ions, which are utilized as redox mediators, have the advantage of undergoing oxidation and reduction reactions easily even at low voltages. In addition, since the oxidation reaction occurs only in ionic form without generating by-products such as gases, it contributes to the safety of the system.

The ferrocyanide may be included in the hydrogel electrolyte layer in an amount of 0.01 M to 0.5 M, and preferably in an amount of 0.05 M to 0.3 M.

The electrolyte layer may further include an additional salt, and in one example, may further include 1 M sodium chloride.

According to one embodiment, the ion patch may further include: a plate-shaped frame; an electrode layer laminated on the frame and including electrodes disposed apart from each other; and gel layers laminated on each of the electrodes.

In another embodiment, the ion patch may be designed without a plate-shaped frame, and may include: an electrode layer including separate anode and cathode electrodes; and gel layers laminated on each of the electrodes.

According to one embodiment, the electrode layer may include an anode comprising Mg and a cathode comprising MoO₃.

In this case, electrons generated from the anode may be supplied to the electrochromic material layer of the electrochromic system to induce an electrochromic reaction of the electrochromic material (e.g., WO₃), while at the same time, at the counter electrode, electrons released from the ferrocyanide ions may move toward the cathode, causing a reduction reaction.

The concentration of the gel may range from 2 to 10 wt %, and preferably from 2 to 8 wt %, 2 to 6 wt %, 2 to 4 wt %, 4 to 10 wt %, or 4 to 8 wt %; for example, it may be from 4 to 6 wt %. However, the concentration is not limited to these ranges.

In one embodiment, the cathode may additionally include a coating material comprising molybdenum (Mo) or titanium (Ti) on the contact surface with the frame.

The coating material may have a thickness of 5 to 30 μm, and preferably a thickness of 5 to 25 μm, 5 to 20 μm, 5 to 15 μm, 10 to 30 μm, 10 to 25 μm, or 10 to 20 μm; for example, it may have a thickness of 10 to 15 μm, but is not limited thereto.

The cathode and anode may each independently have a thickness of 50 to 400 μm.

Preferably, the cathode may have a thickness of 50 to 350 μm, 50 to 300 μm, 100 to 400 μm, 100 to 350 μm, 100 to 300 μm, 150 to 400 μm, 150 to 350 μm, 150 to 300 μm, 200 to 400 μm, or 200 to 350 μm; for example, it may have a thickness of 200 to 300 μm, but is not limited thereto.

Also preferably, the anode may have a thickness of 50 to 350 μm, 50 to 300 μm, 50 to 200 μm, 50 to 150 μm, 100 to 400 μm, 100 to 350 μm, 100 to 300 μm, 100 to 250 μm, or 100 to 200 μm; for example, it may have a thickness of 100 to 150 μm, but is not limited thereto.

The gel layer may include one or more selected from the group consisting of alginate, gelatin, agar, maltose, and glycerol.

In the present invention, the gel laminated on each of the respective electrodes may independently have a thickness of 0.4 to 2 mm, and preferably a thickness of 0.4 to 1.5 mm, 0.4 to 1.2 mm, 0.4 to 1 mm, 0.8 to 2 mm, 0.8 to 1.5 mm, or 0.8 to 1.2 mm; for example, it may have a thickness of 0.8 to 1 mm, but is not limited thereto.

The gel laminated on the cathode may be a drug-loaded gel containing a loaded drug. The loaded drug may include one or more selected from the group consisting of caffeine, niacinamide, and adenosine, but is not limited thereto.

The gel laminated on the anode may be a buffering gel including a pH adjuster. The pH adjuster may be citric acid, but is not limited thereto.

According to one embodiment, the frame may be made of PLGA (polylactic-co-glycolic acid) or PGLA (N,N′-diacetylbacillosaminyl-diphospho-undecaprenol alpha-1,3-N-acetylgalactosaminyltransferase).

According to one embodiment, both the iontophoresis ion patch and the electrochromic unit may be composed of biodegradable materials.

In one embodiment, the inventors configured the ion patch and the electrochromic unit using only biodegradable materials, considering product commercialization for single-use and environmentally friendly disposal.

Various structures of biodegradable microcurrent ion patches are known, and in addition, the inventors introduced tungsten trioxide (WO₃) into the electrochromic layer and a NaCl-based agar gel composition into the electrolyte layer. These components are all well known as biodegradable materials. Furthermore, ferricyanide ions were used for the counter electrode reaction in the electrochromic system, which are also known as redox mediators used in biosensor applications.

According to one embodiment, a resistor connected in series may be further provided between the electrochromic unit and the iontophoresis ion patch.

According to one embodiment, a plurality of electrochromic units may be provided, forming rows arranged in series with each other.

According to one embodiment, a plurality of such rows may be provided, and the rows may be connected in parallel with the iontophoresis ion patch, forming columns.

The inventors developed a technique for visually confirming the amount and usage of drug delivery in the biodegradable microcurrent ion patch by connecting the electrochromic units in various configurations to form an array.

In one embodiment, the current flowing between the electrodes of the ion patch was utilized in the electrochromic units. Four parallel circuits were constructed, each consisting of a row of multiple electrochromic units, with a resistor and an electrochromic unit connected in each circuit. The resistance values were varied for each circuit.

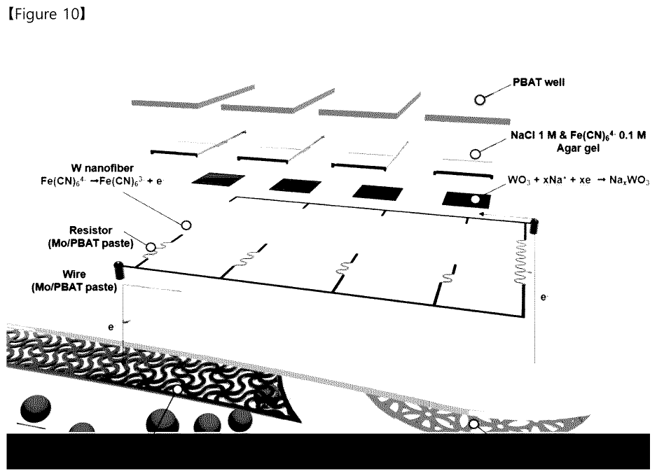

By adjusting the amount of current flowing through each circuit, the four electrochromic units sequentially changed color over time, enabling visual confirmation of the extent of subcutaneous drug delivery. This structure is illustrated in FIGS. 9 and 10.

FIGS. 9 and 10 are merely examples showing the degree of electrochromism, and the electrochromic units can be utilized in various combinations to represent the amount of drug delivery and usage time. Through this configuration, side effects caused by overuse by the user can be prevented, the reliability of the ion patch can be improved, and effective drug delivery or cosmetic application can be achieved.

In the experimental examples shown in FIGS. 9 and 10, conducted by the inventors, the actual current flowing through the conventional and electrode-biodegradable ion patches was approximately 200 μA. When resistors of varying values, ranging from a short circuit to 100 kΩ, were connected to the four parallel circuits, it was confirmed that the current was divided among the circuits. In addition, it was observed that the electrochromic reactions occurred sequentially in each of the four circuits.

The inventors adjusted the resistance values of each circuit to control the amount of current flowing through them and, consequently, the timing of the electrochromic response. As a result, it was confirmed that once the reaction was completed in the electrochromic unit through which the most current initially flowed, the current ceased to flow through that unit and began to flow through the remaining units in which the reaction had not yet been completed, thereby allowing the reactions to proceed sequentially.

In the experimental examples of FIGS. 9 and 10, the optimal usage time of the iontophoresis ion patch was designed by customizing the resistance values such that only two out of the four electrochromic units would be activated.

A method for manufacturing a microcurrent ion patch comprising an electrochromic unit according to another embodiment of the present invention may include:
a step of preparing an electrochromic unit comprising an electrochromic material layer;
a step of preparing an ion patch comprising an anode and a cathode; and
a step of electrically connecting the ion patch and the electrochromic unit.

According to one embodiment, the step of electrically connecting may include connecting one end of the electrochromic material layer to the anode of the ion patch, and connecting the other end of the electrochromic material layer to the cathode of the ion patch, so that electrons emitted from the ion patch pass through the electrochromic unit and re-enter the ion patch.

According to one embodiment, the step of preparing the electrochromic unit may include:
a step of forming the electrochromic material layer on a base thin film; and
a step of forming a hydrogel electrolyte layer on the electrochromic material layer.

The base thin film may include, for example, PBAT.

According to one embodiment, the step of preparing the ion patch may include:
a step of forming an electrode layer by laminating electrodes on a plate-shaped frame; and
a step of forming a gel layer by laminating gel on the electrodes.

The above description is merely illustrative of the technical idea of the present invention. It will be apparent to those of ordinary skill in the art to which the present invention pertains that various modifications and alterations can be made without departing from the essential characteristics of the present invention. Accordingly, the embodiments disclosed herein are intended to explain rather than limit the technical spirit of the present invention, and the scope of the present invention is not limited by these embodiments.

The scope of protection of the present invention should be interpreted based on the following claims, and all technical ideas equivalent thereto should be construed as falling within the scope of the present invention.

1. A microcurrent ion patch comprising:
an iontophoresis ion patch including both electrodes; and
an electrochromic unit electrically connected to the both electrodes,
wherein the electrochromic unit comprises:
a base thin film;
an electrochromic material layer formed on the base thin film; and
a hydrogel electrolyte layer formed on the electrochromic material layer.

2. The microcurrent ion patch according to claim 1,
wherein the electrochromic unit receives electrons from one electrode of the ion patch and supplies the electrons to the other electrode of the ion patch.

3. The microcurrent ion patch according to claim 1,
wherein the electrochromic material layer undergoes a reduction reaction upon receiving electrons, thereby causing electrochromism.

4. The microcurrent ion patch according to claim 1,
wherein the electrochromic unit increases in the area showing color change in correspondence with the amount of drug delivered by the iontophoresis ion patch.

5. The microcurrent ion patch according to claim 1,
wherein the electrochromic material layer includes spaced-apart metal portions and an oxide of the metal filling the spaces between the metal portions.

6. The microcurrent ion patch according to claim 5,
wherein the metal portions comprise tungsten, and the electrochromic material layer comprises tungsten trioxide (WO₃).

7. The microcurrent ion patch according to claim 1,
wherein a redox reaction occurs in the electrolyte layer between ferrocyanide and ferricyanide (Fe(CN)₆⁴⁻/Fe(CN)₆³⁻).

8. The microcurrent ion patch according to claim 1,
wherein the ion patch further comprises:
a plate-shaped frame;
an electrode layer laminated on the frame and including electrodes spaced apart from each other; and
gel layers laminated on each of the electrodes.

9. The microcurrent ion patch according to claim 8,
wherein the electrode layer comprises an anode including Mg and a cathode including MoO₃,
and the gel layer comprises at least one selected from the group consisting of alginate, gelatin, agar, maltose, and glycerol.

10. The microcurrent ion patch according to claim 8,
wherein the frame is made of PLGA (polylactic-co-glycolic acid) or PGLA (N,N′-diacetylbacillosaminyl-diphospho-undecaprenol alpha-1,3-N-acetylgalactosaminyltransferase).

11. The microcurrent ion patch according to claim 1,
wherein both the iontophoresis ion patch and the electrochromic unit are composed entirely of biodegradable materials.

12. The microcurrent ion patch according to claim 1,
further comprising a resistor arranged in series between the electrochromic unit and the iontophoresis ion patch.

13. The microcurrent ion patch according to claim 1,
wherein the electrochromic unit comprises a plurality of units electrically connected in series to form a row.

14. The microcurrent ion patch according to claim 13,
wherein a plurality of the rows are provided, and the rows are connected in a parallel structure with the iontophoresis ion patch to form columns.

15. A method for manufacturing a microcurrent ion patch comprising an electrochromic unit, the method comprising:
a step of preparing an electrochromic unit comprising an electrochromic material layer;
a step of preparing an ion patch comprising an anode and a cathode; and
a step of electrically connecting the ion patch and the electrochromic unit.

16. The method for manufacturing a microcurrent ion patch comprising an electrochromic unit according to claim 15,
wherein the step of electrically connecting comprises:
connecting one end of the electrochromic material layer to the anode of the ion patch, and connecting the other end of the electrochromic material layer to the cathode of the ion patch,
such that electrons emitted from the ion patch pass through the electrochromic unit and re-enter the ion patch.

17. The method for manufacturing a microcurrent ion patch comprising an electrochromic unit according to claim 15,
wherein the step of preparing the electrochromic unit comprises:
a step of forming an electrochromic material layer on a base thin film; and
a step of forming a hydrogel electrolyte layer on the electrochromic material layer.

18. The method for manufacturing a microcurrent ion patch comprising an electrochromic unit according to claim 15,
wherein the step of preparing the ion patch comprises:
a step of forming an electrode layer by laminating electrodes on a plate-shaped frame; and
a step of forming a gel layer by laminating gel on the electrodes.
